(19) United States
(12) Patent Application Publication
(10) Pub. No.: US 2026/0175037 A1
(43) Pub. Date: Jun. 25, 2026

(54) Implantable Neurostimulator Device

(71) Applicant: SecondWave Systems, Inc, Saint Paul, MN (US)

(72) Inventors: Anuj Bhardwaj, Austin, TX (US); Jeffrey Heyman, Somerville, MA (US); Mikel Langron, State College, PA (US); Kashyap Patel, Hoboken, NJ (US); Michael Whetzel, Port Matilda, PA (US)

(21) Appl. No.: 19/125,827

(22) PCT Filed: Nov. 3, 2023
(86) PCT No.: PCT/US2023/078575

§ 371 Date: Apr. 30, 2025

Related U.S. Application Data
(60) Provisional application No. 63/422,478, filed on Nov. 4, 2022.

(51) Int. Cl. A61N 1/372; A61N 1/05

(52) U.S. Cl. CPC A61N 1/37217; A61N 1/05

(57) ABSTRACT

Devices and systems for providing neurostimulation to a target location, may include an implantable neurostimulator device including a first transducer device that includes a gas matrix piezoelectric (GMP) array or a piezoelectric micromachined ultra-sound transducer (pMUT) array; the implantable neurostimulator device has a length of 5 mm to 20 mm and a diameter of 1 mm to 5 mm and an energy delivery device including a second transducer device; the second transducer device includes a gas matrix piezoelectric (GMP) array, a polymer matrix piezocomposite array, a capacitive micro-machined acoustic transducer (cMUT) array, or a pMUT ar-2024/097917 ray; and a beam-forming processor to cause the second transducer device to produce an acoustic wave, the implantable neurostimulator device is to receive the acoustic wave from the energy delivery device, convert the acoustic wave to an electrical energy signal, and apply the electrical energy signal.

### CROSS REFERENCE TO RELATED APPLICATION

This application claims priority to United States Provisional Ser. No. 63/422,478 , filed Nov. 4, 2022, the disclosure of which is hereby incorporated by reference in its entirety.

### BACKGROUND

### Field

The present disclosure relates to implantable medical devices and use of the same for treating patients. More particularly, the present disclosure relates to implantable neurostimulator devices and use of the same for treating various ailments and conditions in patients.

### Technical Considerations

An implantable medical device may refer to an instrument that is either wholly or partially introduced into a body of a patient during a medical procedure. In some instances, physicians implant the implantable medical device during a surgical procedure. However, unlike surgical medical devices, implantable medical devices stay in the body after the medical procedure. In addition, externally powered and/or controlled implantable medical devices may allow for increased comfort and reduced risk of further complications (e.g., infection) for patients as treatments and are provided via the implantable medical devices.

However, providing power and/or control signals may be difficult based on a composition of the body of the patient. For example, providing power and/or control signals to an implantable medical device may be difficult if the implantable medical device is positioned deep inside the body of the patient. Further, parameters for operating an implantable medical device may be limited based on characteristics of the size, construction materials, and/or desired positions in the body of the implantable medical device.

### SUMMARY

Accordingly, disclosed are devices, systems, methods, and products for providing a treatment to a target location, such as an electrical stimulation to a nerve and/or a neuron, using an implantable neurostimulator device.

Further embodiments or aspects are set forth in the following numbered clauses:

Clause 1: An implantable neurostimulator device comprising: at least one transducer device comprising: a gas matrix piezoelectric (GMP) array; or a piezoelectric micromachined ultrasound transducer (pMUT) array; wherein the at least one transducer device comprises a material, and wherein the material comprises: lead zirconate titanate (PZT); single crystal lead magnesium niobate-lead titanate (PMN-PT); aluminum nitride (AlN); scandium (Sc) doped AlN; or any combination thereof; wherein the implantable neurostimulator device has a length in a range of 5 mm to 20 mm and a diameter in a range of 1 mm to 5 mm.

Clause 2: The implantable neurostimulator device of clause 1, wherein the at least one transducer device comprises a plurality of transducer elements, and wherein each transducer element of the plurality of transducer elements comprises: PZT; polyvinylidene fluoride; polyvinylidene difluoride (PVDF); or any combination thereof.

Clause 3: The implantable neurostimulator device of any of clause 1 or clause 2, wherein the plurality of transducer elements are formed in a plurality of rows, and wherein the plurality of rows are spaced equidistant around a perimeter of the at least one transducer device.

Clause 4: The implantable neurostimulator device of any of clauses 1-3, wherein the at least one transducer device comprises the pMUT array, and wherein the pMUT array comprises: PZT; and AlN.

Clause 5: The implantable neurostimulator device of any of clauses 1-4, wherein the at least one transducer device is operated independent of an onboard processor.

Clause 6: The implantable neurostimulator device of any of clauses 1-5, further comprising: an enclosure, wherein the enclosure has a cylindrical design with a plurality of internal surfaces; and wherein the at least one transducer device comprises a plurality of elements of the pMUT array that are positioned on the plurality of internal surfaces of the enclosure.

Clause 7: The implantable neurostimulator device of any of clauses 1-6, wherein the plurality of elements of the pMUT array comprises a plurality of thin film elements deposited on the plurality of internal surfaces of the enclosure.

Clause 8: The implantable neurostimulator device of any of clauses 1-7, wherein the at least one transducer device is configured to: receive an acoustic signal from an energy delivery device; and convert the acoustic signal to an electrical signal to provide an action potential to activate a nerve.

Clause 9: The implantable neurostimulator device of any of clauses 1-8, further comprising: an electrode device, wherein the electrode device is configured to receive the electrical signal and apply the electrical signal to an environment inside a body of a patient.

Clause 10: The implantable neurostimulator device of any of clauses 1-9, further comprising: a rectification circuit, wherein the rectification circuit is configured to covert an output of the at least one transducer to a direct current (DC) electrical pulse to provide electrical stimulation to an environment inside a body of a patient.

Clause11: The implantable neurostimulator device of any of clauses 1-10, wherein the at least one transducer device comprises a plurality of transducer elements, and the implantable neurostimulator device further comprises: a core structure, wherein the core structure is sized and configured to hold the plurality of transducer elements.

Clause 12: A system for providing neurostimulation to a target location of a body of a user, comprising: an implantable neurostimulator device comprising: at least one first transducer device comprising: a gas matrix piezoelectric (GMP) array; or a piezoelectric micromachined ultrasound transducer (pMUT) array; wherein the at least one first transducer device comprises a material, and wherein the material comprises: lead zirconate titanate (PZT); single crystal lead magnesium niobate-lead titanate (PMN-PT); aluminum nitride (AlN); scandium (Sc) doped AlN; or any combination thereof; wherein the implantable neurostimulator device has a length in a range of 5 mm to 20 mm and a diameter in a range of 1 mm to 5 mm; an energy delivery device comprising: at least one second transducer device, wherein the at least one second transducer device comprises a material, and wherein the material comprises: lead zirconate titanate (PZT); polyvinylidene difluoride (PVDF); aluminum nitride (AlN); scandium (Sc) doped AlN; or any combination thereof; wherein the at least one second transducer device comprises: a gas matrix piezoelectric (GMP) array; a polymer matrix piezocomposite array; a capacitive micro-machined acoustic transducer (cMUT) array; or a piezoelectric micromachined ultrasound transducer (pMUT) array; and at least one beam-forming processor configured to: cause the at least one second transducer device to produce an acoustic wave; wherein the implantable neurostimulator device is configured to: receive the acoustic wave from the energy delivery device; covert the acoustic wave to an electrical energy signal; and apply the electrical energy signal to an environment inside a body of a patient.

Clause 13: The system of clause 12, wherein the acoustic wave comprises a plurality of pulses having a pulse repetition frequency, wherein a pulse width of each pulse of the plurality of pulses is in a range of 10 ns to 10 μs, and wherein the pulse repetition frequency is in a range of 1 Hz to 50 Hz.

Clause 14: The system of clause 12 or clause 13, wherein the at least one first transducer device comprises a plurality of transducer elements, and wherein each transducer element of the plurality of transducer elements comprises: PZT; polyvinylidene fluoride; PVDF; or any combination thereof.

Clause 15: The system of any of clauses 12-14, wherein the plurality of transducer elements are formed in a plurality of rows, and wherein the plurality of rows are spaced equidistant around a perimeter of the at least one first transducer device.

Clause 16: The system of any of clauses 12-15, wherein the at least one first transducer device comprises the pMUT array, and wherein the pMUT array comprises: PZT; and AlN.

Clause 17: The system of any of clauses 12-16, wherein the at least one first transducer device is operated independent of an onboard processor.

Clause 18: The system of any of clauses 12-17, further comprising: an enclosure, wherein the enclosure has a cylindrical design with a plurality of internal surfaces; and wherein the at least one first transducer device comprises a plurality of elements of the pMUT array that are positioned on the plurality of internal surfaces of the enclosure.

Clause 19: The system of any of clauses 12-18, wherein the plurality of elements of the pMUT array comprises a plurality of thin film elements deposited on the plurality of internal surfaces of the enclosure.

Clause 20: The system of any of clauses 12-19, wherein the at least one first transducer device is configured to: receive an acoustic signal from an energy delivery device; and convert the acoustic signal to an electrical signal to provide an action potential to activate a nerve.

Clause 21: The system of any of clauses 12-20, further comprising: an electrode device, wherein the electrode device is configured to receive the electrical signal and apply the electrical signal to an environment inside a body of a patient.

Clause 22: The system of any of clauses 12-21, further comprising: a rectification circuit, wherein the rectification circuit is configured to covert an output of the at least one first transducer to a direct current (DC) electrical pulse to provide electrical stimulation to an environment inside a body of a patient.

Clause 23: The system of any of clauses 12-22, wherein the at least one first transducer device comprises a plurality of transducer elements, and the implantable neurostimulator device further comprises: a core structure, wherein the core structure is sized and configured to hold the plurality of transducer elements.

Clause 24: A method of treating pain in a patient, comprising: stimulating a nerve or neuron in the patient with an implantable neurostimulator device, wherein implantable neurostimulator device comprises: at least one transducer device, wherein the at least one transducer device comprises a material, and wherein the material comprises: lead zirconate titanate (PZT); single crystal lead magnesium niobate-lead titanate (PMN-PT); aluminum nitride (AlN); scandium (Sc) doped AlN; or any combination thereof; wherein the at least one transducer device comprises: a gas matrix piezoelectric (GMP) array; or a piezoelectric micromachined ultrasound transducer (pMUT) array; wherein the implantable neurostimulator device has a length in a range of 5 mm to 20 mm and a diameter in a range of 1 mm to 5 mm; and wherein the nerve or neuron is one or more of supratrochlear, auriculotemporal, orbital, vagus, trigeminal, dorsal root ganglion, and/or sphenopalatine ganglion nerve or neuron; thereby treating the pain in the patient.

Clause 25: The method of clause 24, wherein stimulating the nerve or neuron with an implantable neurostimulator device, comprises: stimulating one or more branches of the trigeminal nerve, wherein the one or more branches of the trigeminal nerve comprises ophthalmic, maxillary, mandibular, supraorbital, infraorbital, occipital, and/or auriculotemporal branches of the trigeminal nerve.

Clause 26: The method of clause 24 or clause 25, wherein the pain comprises a migraine headache or a cluster headache.

Clause 27: The method of any of clauses 24-26, wherein the pain comprises craniofacial pain.

Clause 28: The method of any of clauses 24-27, wherein the pain comprises head and/or neck pain.

Clause 29: The method of any of clauses 24-28, wherein the pain comprises regional pain, wherein the reginal pain comprises chronic regional pain syndrome.

Clause 30: A method of treating a condition associated with inflammation and/or immunoregulation in a patient, comprising: stimulating a nerve or a neuron in the patient with an implantable neurostimulator device, wherein the implantable neurostimulator device comprises: at least one transducer device, wherein the at least one transducer device comprises a material, and wherein the material comprises: lead zirconate titanate (PZT); single crystal lead magnesium niobate-lead titanate (PMN-PT); aluminum nitride (AlN); scandium (Sc) doped AlN; or any combination thereof; wherein the at least one transducer device comprises: a gas matrix piezoelectric (GMP) array; or a piezoelectric micromachined ultrasound transducer (pMUT) array; wherein the implantable neurostimulator device has a length in a range of 5 mm to 20 mm and a diameter in a range of 1 mm to 5 mm; and wherein the nerve or the neuron is a splenic nerve or neuron; thereby treating the condition associated with inflammation and/or immunoregulation in the patient.

Clause 31: The method of clause 30, wherein the condition associated with inflammation and/or immunoregulation comprises: inflammatory arthritis, inflammatory bowel disease, multiple sclerosis, post-surgical inflammation, and/or post-ischemic inflammation.

Clause 32: The method of clause 30 or clause 31, wherein the condition associated with inflammation and/or immunoregulation comprises rheumatoid arthritis, ankylosing spondylitis, psoriatic arthritis, and/or lupus.

Clause 33: A method of treating hypertension in a patient, comprising: stimulating an artery in the patient with an implantable neurostimulator device, wherein the implantable neurostimulator device comprises: at least one transducer device, wherein the at least one transducer device comprises a material, and wherein the material comprises: lead zirconate titanate (PZT); single crystal lead magnesium niobate-lead titanate (PMN-PT); aluminum nitride (AlN); scandium (Sc) doped AlN; or any combination thereof; wherein the at least one transducer device comprises: a gas matrix piezoelectric (GMP) array; or a piezoelectric micromachined ultrasound transducer (pMUT) array; and wherein the implantable neurostimulator device has a length in a range of 5 mm to 20 mm and a diameter in a range of 1 mm to 5 mm; thereby treating hypertension in the patient.

Clause 34: The method of clause 33, wherein stimulating the artery in the patient with the implantable neurostimulator device comprises: stimulating a renal artery in the patient with the implantable neurostimulator device.

Clause 35: A method of treating a condition associated with overactive bladder and/or pelvic floor dysfunction in a patient, comprising: stimulating a nerve or a neuron in the patient with an implantable neurostimulator device, wherein the implantable neurostimulator device comprises: at least one transducer device, wherein the at least one transducer device comprises a material, and wherein the material comprises: lead zirconate titanate (PZT); single crystal lead magnesium niobate-lead titanate (PMN-PT); aluminum nitride (AlN); scandium (Sc) doped AlN; or any combination thereof; wherein the at least one transducer device comprises: a gas matrix piezoelectric (GMP) array; or a piezoelectric micromachined ultrasound transducer (pMUT) array; wherein the implantable neurostimulator device has a length in a range of 5 mm to 20 mm and a diameter between 1 mm to 5 mm; and wherein the nerve or neuron is a tibial and/or sacral nerve or neuron; and thereby treating the condition associated with overactive bladder and/or pelvic floor dysfunction in the patient in the patient.

These and other features and characteristics of the presently disclosed subject matter, as well as the methods of operation and functions of the related elements of structures and the combination of parts and economies of manufacture, will become more apparent upon consideration of the following description and the appended claims, all of which form a part of this specification, wherein like reference numerals designate corresponding parts in the various figures. It is to be expressly understood, however, that the drawings are for the purpose of illustration and description only and are not intended as a definition of the limits of the disclosed subject matter. As used in the specification and the claims, the singular form of “a,” “an,” and “the” include plural referents unless the context clearly dictates otherwise.

### BRIEF DESCRIPTION OF THE DRAWINGS

Additional advantages and details are explained in greater detail below with reference to the exemplary embodiments that are illustrated in the accompanying schematic figures, in which:

FIG. 1 is a diagram of a non-limiting embodiment or aspect of an environment in which systems, devices, products, apparatus, and/or methods, described herein, may be implemented according to the principles of the present disclosure;

FIG. 2 is a diagram of a non-limiting embodiment of components of one or more devices shown in FIG. 1;

FIG. 3 is a diagram of a non-limiting embodiment of an implantable neurostimulator device;

FIG. 4 is a diagram of a non-limiting embodiment of an energy delivery device;

FIG. 5 is a diagram of a non-limiting embodiment of a system for providing neurostimulation to a target location of a body of a user;

FIGS. 6A-6G are diagrams of a non-limiting embodiment of an implantable neurostimulator device;

FIG. 7 is a diagram of another non-limiting embodiment of an implantable neurostimulator device; and

FIG. 8 is a diagram of another non-limiting embodiment of an implantable neurostimulator device.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosure as it is oriented in the drawing figures. However, it is to be understood that the disclosure may assume various alternative variations and step sequences, except where expressly specified to the contrary. It is also to be understood that the specific devices and processes illustrated in the attached drawings, and described in the following specification, are simply exemplary embodiments or aspects of the disclosure. Hence, specific dimensions and other physical characteristics related to the embodiments or aspects of the embodiments disclosed herein are not to be considered as limiting unless otherwise indicated.

No aspect, component, element, structure, act, step, function, instruction, and/or the like used herein should be construed as critical or essential unless explicitly described as such. In addition, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. The phrase “based on” may also mean “in response to” where appropriate and may refer to a condition for automatically triggering a specified operation of an electronic device (e.g., a computing device or the like).

Embodiments of the present disclosure may include an implantable neurostimulator device comprising at least one transducer device that includes a gas matrix piezoelectric (GMP) array, or a piezoelectric micromachined ultrasound transducer (pMUT) array, wherein the at least one transducer device comprises a material, wherein the material includes lead zirconate titanate (PZT), single crystal lead magnesium niobate-lead titanate (PMN-PT), aluminum nitride (AlN), scandium (Sc) doped AlN, or any combination thereof, and wherein the implantable neurostimulator device has a length in a range of 5 mm to 20 mm and a diameter in a range of 1 mm to 5 mm. In some non-limiting embodiments, the at least one transducer device comprises a plurality of transducer elements, wherein each transducer element of the plurality of transducer elements includes PZT, polyvinylidene fluoride, polyvinylidene difluoride (PVDF), or any combination thereof. In some non-limiting embodiments, the plurality of transducer elements are formed in a plurality of rows, wherein the plurality of rows are spaced equidistant around a perimeter of the at least one transducer device. In some non-limiting embodiments, the at least one transducer device comprises the pMUT array, wherein the pMUT array includes PZT and AlN. In some non-limiting embodiments, the at least one transducer device is operated independent of an onboard processor. In some non-limiting embodiments, the implantable neurostimulator device includes an enclosure, wherein the enclosure has a cylindrical design with a plurality of internal surfaces, and wherein the at least one transducer device comprises a plurality of elements of the pMUT array that are positioned on the plurality of internal surfaces of the enclosure. In some non-limiting embodiments, the plurality of elements of the pMUT array comprises a plurality of thin film elements deposited on the plurality of internal surfaces of the enclosure. In some non-limiting embodiments, the at least one transducer device is configured to receive an acoustic signal from an energy delivery device and convert the acoustic signal to an electrical signal to provide an action potential to activate a nerve. In some non-limiting embodiments, the implantable neurostimulator device includes an electrode device, wherein the electrode device is configured to receive the electrical signal and apply the electrical signal to an environment inside a body of a patient. In some non-limiting embodiments, the implantable neurostimulator device includes a rectification circuit, wherein the rectification circuit is configured to covert an output of the at least one transducer to a DC electrical pulse to provide electrical stimulation to an environment inside a body of a patient. In some non-limiting embodiments, the at least one transducer device includes a plurality of transducer elements, and the implantable neurostimulator device includes a core structure, wherein the core structure is sized and configured to hold the plurality of transducer elements.

In some non-limiting embodiments, a system for providing neurostimulation to a target location of a body of a user, includes an implantable neurostimulator device comprising at least one transducer device that includes a gas matrix piezoelectric (GMP) array, or a piezoelectric micromachined ultrasound transducer (pMUT) array, wherein the at least one transducer device comprises a material, wherein the material includes lead zirconate titanate (PZT), single crystal lead magnesium niobate-lead titanate (PMN-PT), aluminum nitride (AlN), scandium (Sc) doped AlN, or any combination thereof, and wherein the implantable neurostimulator device has a length in a range of 5 mm to 20 mm and a diameter in a range of 1 mm to 5 mm. In some non-limiting embodiments, the system includes an energy delivery device that includes at least one second transducer device, wherein the at least one second transducer device comprises a material, and wherein the material includes PZT, PVDF, AlN, scandium (Sc) doped AlN, any combination thereof. In some non-limiting embodiments, the at least one second transducer device includes a GMP array, a polymer matrix piezocomposite array, a capacitive micro-machined acoustic transducer (cMUT) array, or a piezoelectric micromachined ultrasound transducer (pMUT) array. In some non-limiting embodiments, the energy delivery device includes at least one beam-forming processor configured to cause the at least one second transducer device to produce an acoustic wave, wherein the implantable neurostimulator device is configured to receive the acoustic wave from the energy delivery device, covert the acoustic wave to an electrical energy signal, and apply the electrical energy signal to an environment inside a body of a patient. In some non-limiting embodiments, the acoustic wave comprises a plurality of pulses having a pulse repetition frequency, wherein a pulse width of each pulse of the plurality of pulses is in a range of 10 ns to 10 μs, and wherein the pulse repetition frequency is in a range of 1 Hz to 50 Hz.

In this way, embodiments of the present disclosure provides an implantable neurostimulator device and a system for providing neurostimulation to a target location of a body of a user that allows for providing power and/or control signals to the implantable medical device that is positioned inside the body of the patient. Further, embodiments of the present disclosure provide an implantable medical device that allows for accurate and configurable operation to provide neurostimulation to a target location that also has a form factor that allows for implantation in any location inside the body of the patient.

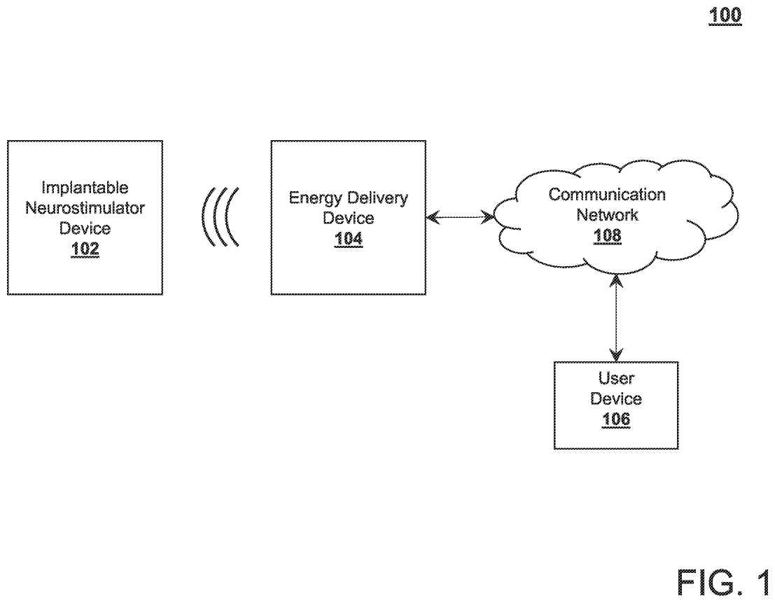

Referring now to FIG. 1, FIG. 1 is a diagram of an example environment 100 in which devices, systems, methods, and/or products described herein may be implemented. As shown in FIG. 1, environment 100 includes implantable neurostimulator device 102, energy delivery device 104, user device 106, and communication network 108. In some non-limiting embodiments, energy delivery device 104 and user device 106 may interconnect (e.g., establish a connection to communicate, establish a communication connection, etc.) via wired connections, wireless connections, or a combination of wired and wireless connections.

Implantable neurostimulator device 102 may include one or more devices configured to receive an acoustic signal from an energy delivery device, convert the acoustic signal to an electrical signal to provide an action potential to activate a nerve, and/or apply the electrical signal to an environment inside a body of a user. Implantable neurostimulator device 102 may have a form factor that allows for implantation inside a body of a user. For example, implantable neurostimulator device 102 may be sized and configured to be implantable at a specific location inside the body of a user. In some non-limiting embodiments, implantable neurostimulator device 102 may have a length in a range of 5 mm to 20 mm and a width (e.g., a diameter) in a range of 1 mm to 5 mm.

Energy delivery device 104 may include one or more devices configured to provide power, in the form of acoustic waves (e.g., ultrasonic waves, ultrasonic beams, etc.), that is to be received by implantable neurostimulator device 102 (e.g., that is implanted inside a body of a user). For example, energy delivery device 104 may include appropriate electrical circuit components, such as a transducer and a processor to control the transducer. In some non-limiting embodiments, energy delivery device 104 may be sized and configured to be wearable on the body of the user. For example, energy delivery device 104 may have a wearable form-factor that allows for energy delivery device 104 to be placed upon and/or adhered to a body of a patient (e.g., a torso of a patient) for a period of time. In some non-limiting embodiments, energy delivery device 104 may have the following dimensions, including a length in a range of 60 to 250 mm, a width in a range of 40 to 250 mm, and a height in a range of 15 to 50 mm. In one example, energy delivery device 104 may have the following dimensions a length of 75 mm, a width of 55 mm, and a height of 19 mm. In some non-limiting embodiments, energy delivery device 104 may include a power source that is carried on board. For example, energy delivery device 104 may include a battery having a capacity in a range of 50 to 900,000 mAh.

In some non-limiting embodiments, energy delivery device 104 may include a 32-channel phased transducer array, which may be capable of providing an equivalent of 128-channel beam forming capability for high resolution focusing by mirroring the phased transducer array design on two different axes. In such an example, energy delivery device 104 may provide a peak frequency at 400 kHz and channel spacing at 1.5 mm, which may be equivalent to less than half of an ultrasonic wavelength in water and provides the ability to steer and focus an acoustic wave by avoiding energy loss due to side lobe generation. In some non-limiting embodiments, energy delivery device 104 may include circuitry and a transducer device capable of steering and focusing an acoustic wave with center focus conditions with target depths between 1 and 500 mm, steering conditions with target depths between 1 and 500 mm, and steering angles between 0 and 90 degrees in a volumetric space.

In some non-limiting embodiments, energy delivery device 104 may provide power (e.g., as an acoustic signal) to implantable neurostimulator device 102 based on a transducer of energy delivery device 104 that has channel control. In some non-limiting embodiments, implantable neurostimulator device 102 may be powered by a 2-dimensional phased array ultrasonic transducer with 32-channel control of energy delivery device 104. In some non-limiting embodiments, implantable neurostimulator device 102 may be powered by a 2-dimensional phased array ultrasonic transducer with 128-channel control of energy delivery device 104. In some non-limiting embodiments, implantable neurostimulator device 102 may be powered by a 2-dimensional phased array ultrasonic transducer with 256 ultrasonic piezoelectric elements of energy delivery device 104. In this way, energy delivery device 104 may control individual transducer elements of a transducer device of Implantable neurostimulator device 102. Further details regarding embodiments of energy delivery device 104 may be found in International Patent Application No. PCT/US2021/030464 filed on May 3, 2021, which is incorporated by reference herein in its entirety.

User device 106 may include one or more devices configured to be in communication with energy delivery device 104 via communication network 108. For example, user device 106 may include a desktop computer (e.g., a client device that communicates with a server), a mobile computer (e.g., a mobile device, such as a smartphone, a tablet, etc.), and/or the like. User device 106 may be configured to transmit data to and/or receive data from energy delivery device 104 via a short-range wireless communication connection (e.g., a near-field communication (NFC) connection, a radio frequency identification (RFID) communication connection, a Bluetooth® communication connection, and/or the like). In some non-limiting embodiments or aspects, user device 106 may be associated with a user or an individual (e.g., a medical practitioner) administering a treatment with implantable neurostimulator device 102 and energy delivery device 104. In some non-limiting embodiments, user device 106 may include an interface (e.g., a graphical user interface (GUI)) that allows for selection of operational features for control (e.g., only control) of energy delivery device 104 based on an application (e.g., a computer application, a mobile application, etc.).

Communication network 108 may include one or more wired and/or wireless networks. For example, communication network 108 may include a cellular network (e.g., a long-term evolution (LTE) network, a third generation (3G) network, a fourth generation (4G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of some or all of these or other types of networks.

The number and arrangement of devices shown in FIG. 1 are provided as an example. There may be additional devices, fewer devices, different devices, or differently arranged devices than those shown in FIG. 1. Furthermore, two or more devices shown in FIG. 1 may be implemented within a single device, or a single device shown in FIG. 1 may be implemented as multiple devices. Additionally or alternatively, a set of devices of environment 100 may perform one or more functions described as being performed by another set of devices of environment 100.

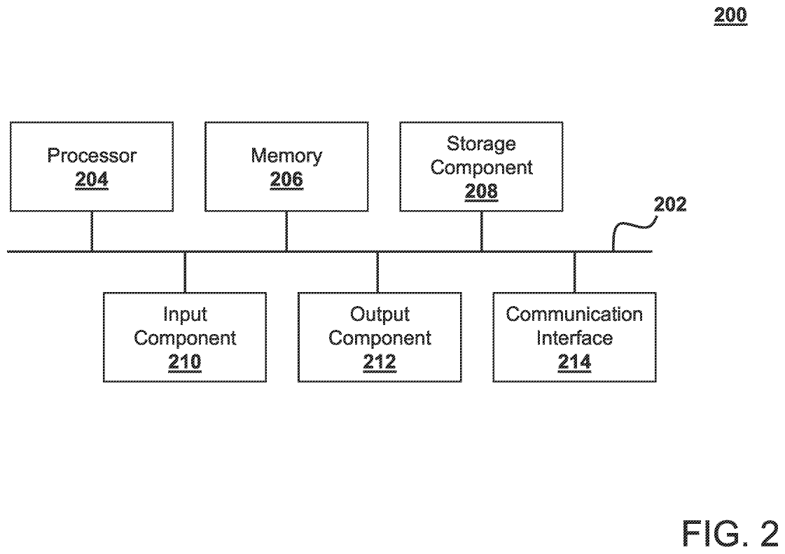

Referring now to FIG. 2, FIG. 2 is a diagram of example components of device 200. Device 200 may correspond to implantable neurostimulator device 102, energy delivery device 104, and/or user device 106. In some non-limiting embodiments or aspects, implantable neurostimulator device 102, energy delivery device 104, and/or user device 106 may include at least one device 200 and/or at least one component of device 200. As shown in FIG. 2, device 200 may include bus 202, processor 204, memory 206, storage component 208, input component 210, output component 212, and communication interface 214. Additionally or alternatively, device 200 may correspond to other devices disclosed herein, including power control module 504 and/or power source 506.

Bus 202 may include a component that permits communication among the components of device 200. In some non-limiting embodiments or aspects, processor 204 may be implemented in hardware or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204. In some non-limiting embodiments, processor 204 may include a beam forming microprocessor as described herein.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a Bluetooth® interface, a Zigbee® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database and/or the like). Device 200 may be capable of receiving information from, storing information in, communicating information to, or searching information stored in the data storage or one or more data structures in memory 206 and/or storage component 208. For example, the information may include input data, output data, transaction data, account data, or any combination thereof.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

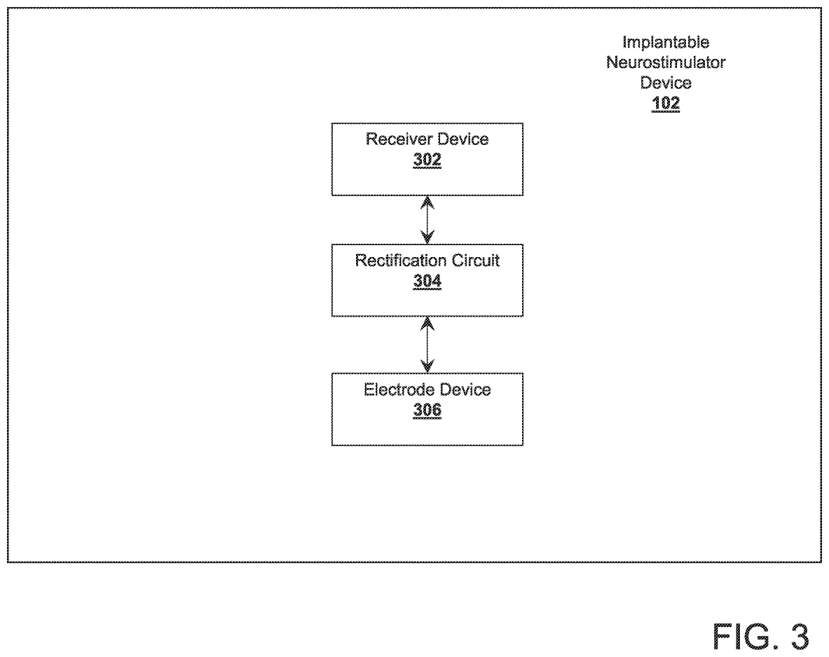

Referring now to FIG. 3, FIG. 3 is a diagram of implantable neurostimulator device 102. As shown in FIG. 3, implantable neurostimulator device 102 may include receiver device 302, rectification circuit 304, and electrode device 306. In some non-limiting embodiments, implantable neurostimulator device 102 may be covered (e.g., encapsulated) with a biocompatible material, such as alumina and/or a material including alumina.

In some non-limiting embodiments, receiver device 302 may include one or more devices that are configured to receive an acoustic signal (e.g., an acoustic wave, a plurality of acoustic waves, etc.) and convert the acoustic signal to an electrical signal (e.g., an electrical current) to provide an action potential to activate a nerve. For example, receiver device 302 may include one or more transducer devices.

In some non-limiting embodiments, the transducer device may include a transducer array, where the transducer array includes a plurality of transducer elements (e.g., individual transducer elements). In some non-limiting embodiments, the plurality of transducer elements may be connected with one or more electrodes on a first side and one or more electrodes on a second side (e.g., a side opposite the first side). In some non-limiting embodiments, the plurality of transducer elements may be controlled with signal channels individually (e.g., each transducer element controlled with one signal channel) or as a plurality (e.g., a set of transducer elements controlled with one signal channel). In one example, a first set of transducer elements of the transducer array may be controlled by a first processor (e.g., a first beam forming microprocessor, such as beam forming microprocessor 402 of energy delivery device 104), and a second set of transducer elements of the transducer array may be controlled by a second processor (e.g., a second beam forming microprocessor, such as another beam forming microprocessor 402 of energy delivery device 104).

In some non-limiting embodiments, the transducer device may be made (e.g., constructed, fabricated, etc.) from a material that may include lead zirconate titanate (PZT), polyvinylidene difluoride (PVDF), aluminum nitride (AlN), scandium (Sc) doped AlN, or a combination of these materials. In some non-limiting embodiments, the transducer device may include a gas matrix piezoelectric (GMP) array, a capacitive micro-machined acoustic transducer (cMUT) array, and/or a piezoelectric micro-machined ultrasound transducer (pMUT) array. In some non-limiting embodiments, the transducer device may include a microelectronic mechanical systems (MEMS) transducer device.

In some non-limiting embodiments, receiver device 302 (e.g., a transducer device of receiver device 302) may be operated independent of an onboard processor (e.g., a processor is not included onboard receiver device 302). For example, receiver device 302 may be constructed without a processor (e.g., a controller, such as a microcontroller) for controlling operations of receiver device 302. In this way, receiver device 302 may be a passive electronic component that receives an acoustic signal and the construction of receiver device 302 may determine how receiver device 302 operates to produce an electrical signal. In some non-limiting embodiments, receiver device 302 may be operated with a processor. For example, individual elements of receiver device 302 (e.g., individual elements of a transducer device of receiver device 302) may be operated based on control signals provided by a processor.

In some non-limiting embodiments, rectification circuit 304 may include one or more devices for converting an alternating current (AC) electrical signal into a direct current (DC) electrical signal. For example, rectification circuit 304 may include one or diodes (e.g., one or more P-N junction diodes). In some non-limiting embodiments, rectification circuit 304 may convert an AC electrical signal into DC pulse for stimulation. In some non-limiting embodiments, rectification circuit 304 may be configured to covert an output of receiver device 302 (e.g., one or more transducer devices of receiver device 302) to a DC electrical pulse to provide electrical stimulation to an environment inside a body of a patient.

In some non-limiting embodiments, rectification circuit 304 may produce an output of up to 10 V and 10 mA (e.g., when electrode device 306 is exposed to resistance of 1 k-ohm). In some non-limiting embodiments, rectification circuit 304 may be configured for rectification of individual transducer elements for lower destructive interference during summation of transducer element channels. In some non-limiting embodiments, rectification circuit 304 may include an application specific integrated circuit (ASIC) to perform a method of signal rectification and/or transformation, which may include channel multiplexing, for single or multi-channel nerve activation (e.g., based on operation of individual transducer elements).

In some non-limiting embodiments, electrode device 306 may include one or more devices for providing a conducting surface to apply an electrical signal (e.g., an AC electrical signal or a DC electrical signal) to a body of a user. For example, electrode device 306 may include one or more electrodes, one or more electrode components, one or more electrical leads, and/or the like. In some non-limiting embodiments, electrode device 306 may be configured to receive an electrical signal (e.g., an electrical signal from rectification circuit 304) and apply the electrical signal to an environment inside a body of a patient. In some non-limiting embodiments, electrode device 306 may be connected to (e.g., electrically connected to) receiver device 302 (e.g., a transducer device of receiver device 302). In some non-limiting embodiments, electrode device 306 may include a conductive material, such as a metal. For example, electrode device 306 may be constructed of a conductive material formed in an appropriate shape. In some non-limiting embodiments, electrode device 306 may include one or more leads that are configured to provide stimulation to a location (e.g., a location of a nerve or neuron) of a body of a user. In some non-limiting embodiments, the lead may include an insulated wire of an appropriate size. In some non-limiting embodiments, electrode device 306 may be formed integrally with receiver device 302.

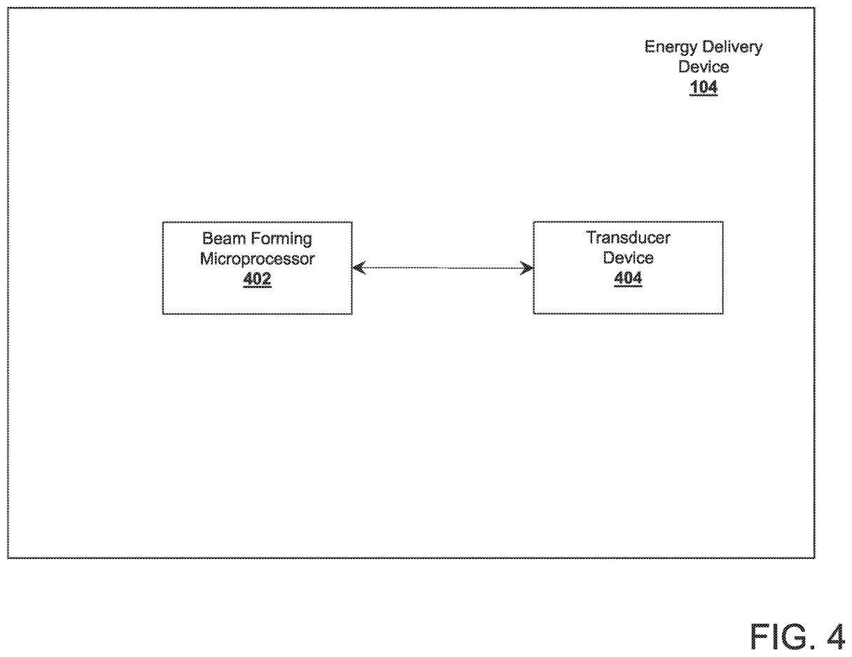

Referring now to FIG. 4, FIG. 4 is a diagram of energy delivery device 104. As shown in FIG. 4, energy delivery device 104 may include beam forming microprocessor 402 and transducer device 404. In some non-limiting embodiments, beam forming microprocessor 402 and transducer device 404 may interconnect (e.g., establish a connection to communicate, establish a communication connection, etc.) via wired connections, wireless connections, or a combination of wired and wireless connections. In some non-limiting embodiments, beam forming microprocessor 402 and transducer device 404 may be connected via a bus (e.g., bus 202).

In some non-limiting embodiments, beam forming microprocessor 402 may include one or more devices that are configured to control transducer device 404. For example, beam forming microprocessor 402 may include a microprocessor that is configured to provide high-voltage, high-frequency electrical signals to transducer device 404 and transducer device 404 may convert the high-voltage, high-frequency electrical signals into an output, where the output may include an acoustic wave. In some non-limiting embodiments, beam forming microprocessor 402 may include a phase delay feature that allows for steering and focusing of acoustic waves produced by transducer device 404.

In some non-limiting embodiments, beam forming microprocessor 402 may include the following specifications: a channel count from 1 to 32,768; electrical current limit per channel from 0.001 to 5 A; zero to peak voltage from 5 to 500V; peak-to-peak voltage from 10 to 1000V; and/or zero to peak negative from −5to −500 V. In some non-limiting embodiments, beam forming microprocessor 402 may include an application specific integrated circuit (ASIC). In some non-limiting embodiments, beam forming microprocessor 402 may have the following dimensions: a length in a range of 50-70 mm; a width in a range of 30-70 mm; and a height in a range of 1-20 mm. In some non-limiting embodiments, beam forming microprocessor 402 may include the following output specifications: voltage signal outputs in a range of 1 to 1,000V peak to peak; a clock frequency up to 500 MHz; and memory to store information, such as waveforms and algorithms (e.g., phasing algorithms for individual transducer device channel control to allow focusing and/or steering of an acoustic wave produced by the transducer device). In some non-limiting embodiments, beam forming microprocessor 402 may include the following input specifications: an interface (e.g., Bluetooth® interface) for receiving signals; on board programming; control of one or more channels; control of one or more channel delays; delay resolution in a range of 1 ns to 500 μs; and a plurality of input/output (I/O) pins for processing various information, including trigger conditions.

In some non-limiting embodiments, transducer device 404 may include one or more devices that are configured to produce an acoustic wave. For example, transducer device 404 may include a transducer that is configured to produce an acoustic wave (e.g., an ultrasonic beam, an ultrasonic wave, etc.) having specific parameters. In some non-limiting embodiments, transducer device 404 may include a transducer that can be used to send and receive signals (e.g., pulse-echo) and/or receive signals transmitted by an energy delivery transducer or another transducer (e.g., pitch-catch).

In some non-limiting embodiments, transducer device 404 may include an energy delivery transducer. For example, transducer device 404 may include an energy delivery transducer that is configured to provide an acoustic wave that may be configured to provide therapy to a patient, which may be used as a detection signal for steering a beam path of the acoustic wave, and/or the like. In some non-limiting embodiments, transducer device 404 may include a detection transducer (e.g., an imaging transducer). For example, transducer device 404 may include a detection transducer that is configured to receive an acoustic wave that is a reflection of an acoustic wave provided by transducer device 404 and provide data associated with the reflection of an acoustic wave. In some non-limiting embodiments, transducer device 404 may be configured to provide data associated with an image (e.g., an ultrasound image) of an object based on the reflection of an acoustic wave.

In some non-limiting embodiments, transducer device 404 may include a transducer array, where the transducer array include includes a plurality of transducer elements (e.g., individual transducer elements). In some non-limiting embodiments, the plurality of transducer elements may be connected with one or more ground plane electrodes on a first side and a signal channel connection on a second side (e.g., a side opposite the first side). In some non-limiting embodiments, the plurality of transducer elements may be controlled with signal channels individually (e.g., each transducer element controlled with one signal channel) or as a plurality (e.g., a set of transducer elements controlled with one signal channel). In one example, a first beam forming microprocessor (e.g., one beam forming microprocessor 402) may control a first set of transducer elements of a transducer array and a second beam forming microprocessor (e.g., one beam forming microprocessor 402) may control a second set of transducer elements of the transducer array.

In some non-limiting embodiments, transducer device 404 may be made (e.g., constructed, fabricated, etc.) from a material that may include lead zirconate titanate (PZT), polyvinylidene difluoride (PVDF), aluminum nitride (AlN), scandium (Sc) doped AlN, or a combination of these materials. In some non-limiting embodiments, transducer device 404 may include a gas matrix piezoelectric (GMP) array, a capacitive micro-machined acoustic transducer (cMUT) array and/or a piezoelectric micro-machined ultrasound transducer (pMUT) array. In some non-limiting embodiments, transducer device 404 may include a microelectronic mechanical systems (MEMS) transducer device.

In some non-limiting embodiments, transducer device 404 may be electrically connected to beam forming microprocessor 402 using a balls grid array (BGA), by being directly bonded via a flip chip, a through-silicon via (TSV), using a push/pull connector, using direct soldering, using wire-bond interconnects, and/or the like. In some non-limiting embodiments, transducer device 404 may be made (e.g., formed, constructed, fabricated, etc.) onto a flexible substrate, such as a flexible printed circuit board (PCB) and/or a flexible electrode, to allow for conformance of energy delivery device 104 to a body of a user.

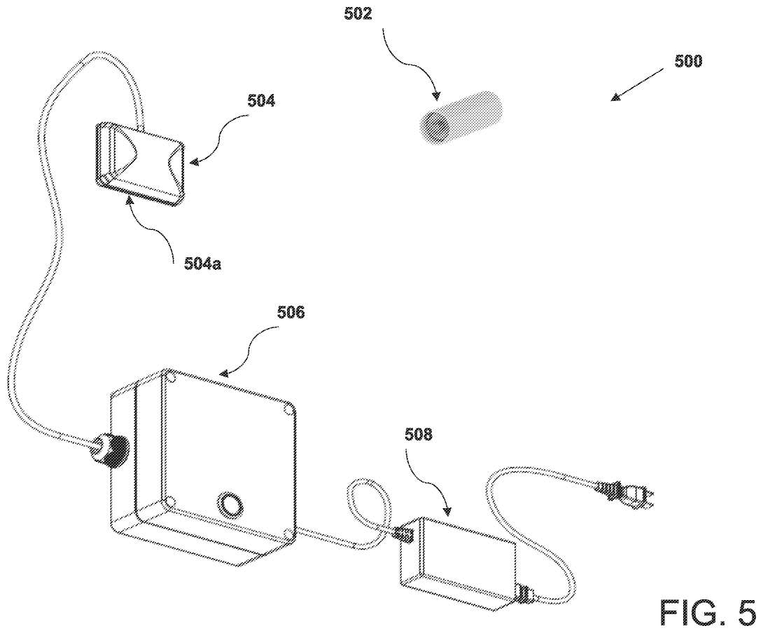

Referring now to FIG. 5, FIG. 5 is a diagram of system 500 for providing neurostimulation to a target location of a body of a user (e.g., a patient, a recipient of treatment, etc.). As shown in FIG. 5, system 500 may include implantable neurostimulator device 502, energy delivery device 504, power control module 506, and power source 508. In some non-limiting embodiments, implantable neurostimulator device 502 may be the same as or similar to implantable neurostimulator device 102. In some non-limiting embodiments, energy delivery device 504 may be the same as or similar to energy delivery device 104.

In some non-limiting embodiments, implantable neurostimulator device 502 is sized and configured to be implanted inside a body of a user and configured to receive an acoustic signal generated by energy delivery device 504. In some non-limiting embodiments, energy delivery device 504 is configured to be applied to an external surface (e.g., on the skin) of the body and to provide the acoustic signal according to predetermined parameters.

In some non-limiting embodiments, energy delivery device 504 may include coupling pad 504a for coupling (e.g., acoustically coupling) energy delivery device 504 to the body of the user. In some non-limiting embodiments, coupling pad 504a may include a material for coupling energy delivery device 504 to a body of a user. For example, coupling pad 504a may include an adhesive, such as a biocompatible adhesive.

In some examples, coupling pad 504a may include a solid coupling material or semi-solid coupling material. The semi-solid coupling material may include a hydrogel having an internal water content that is greater than 20%. In some non-limiting embodiments, the material for coupling may include an adhesive, a liquid-based acoustic coupling gel, and/or the like. In some non-limiting embodiments, coupling pad 504a may be disposable. In some non-limiting embodiments, coupling pad 504a may include a hydrogel material that provides acoustic coupling of energy delivery device 504 to acoustically match energy delivery device 504 to the skin of the user during operation.

In some non-limiting embodiments, power source 506 may include a device which receives and converts standard electrical power inputs. For example, power source 506 may include a power adapter which can accommodate standard electrical power inputs (e.g., a power signal in a range between 100-240V and having a frequency in a range between 50-60 Hz) and provide an amount of power in a range from 5 to 500 W. Power source 506 may include a plug that is configured to be plugged into a standard wall outlet. In some non-limiting embodiments, power source 506 may be configured to convert an AC power signal (e.g., an AC voltage) to a DC power signal (e.g., a DC voltage). In some non-limiting embodiments, power control module 506 may include electronic circuitry that is configured to provide power conditioning for a power signal that is provided by energy delivery device 504.

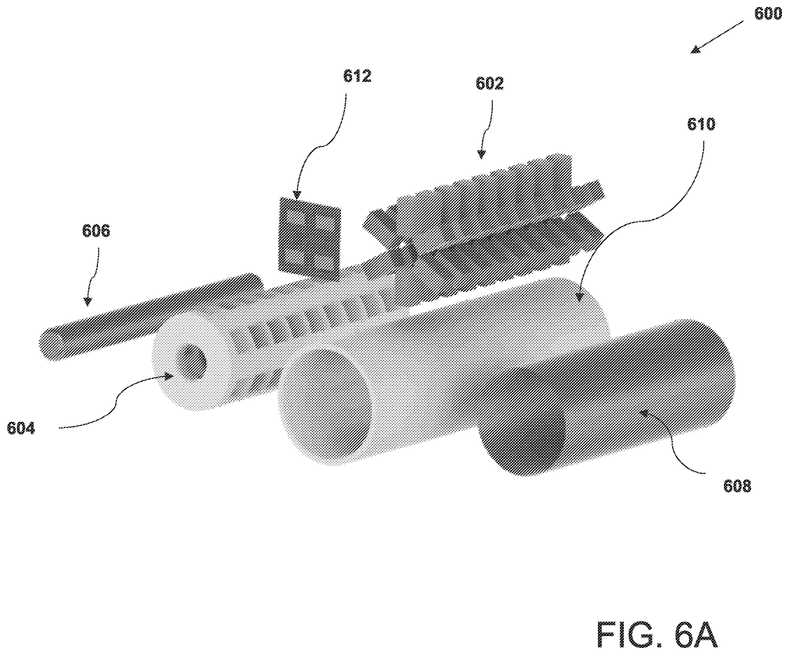

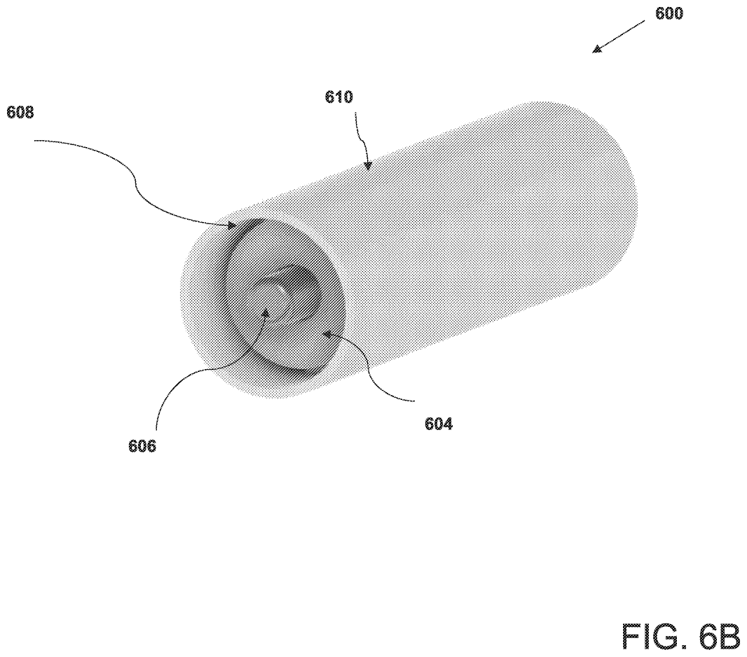

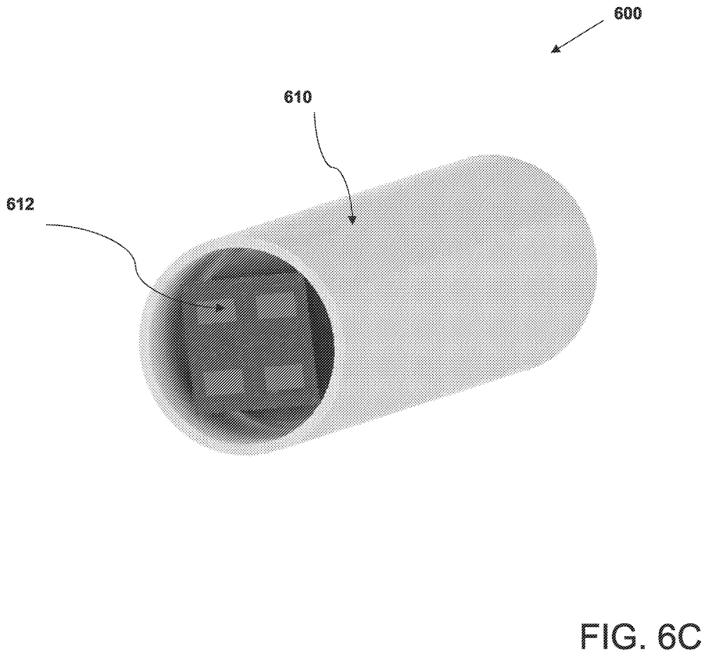

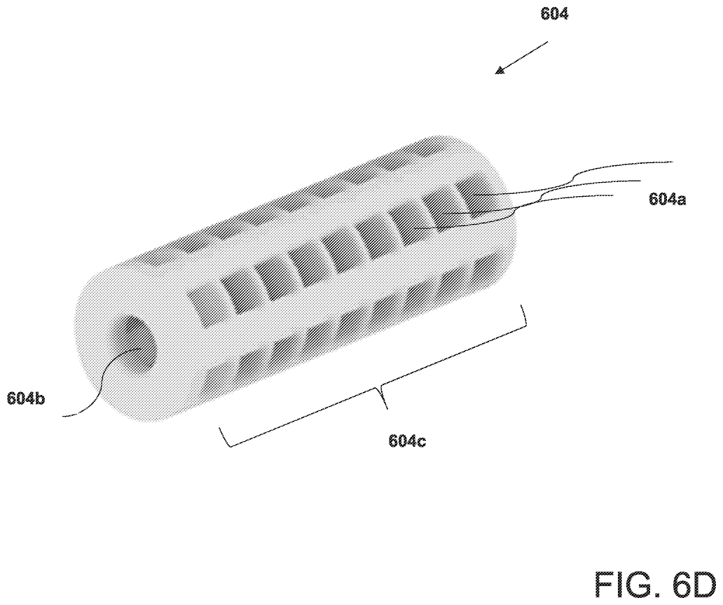

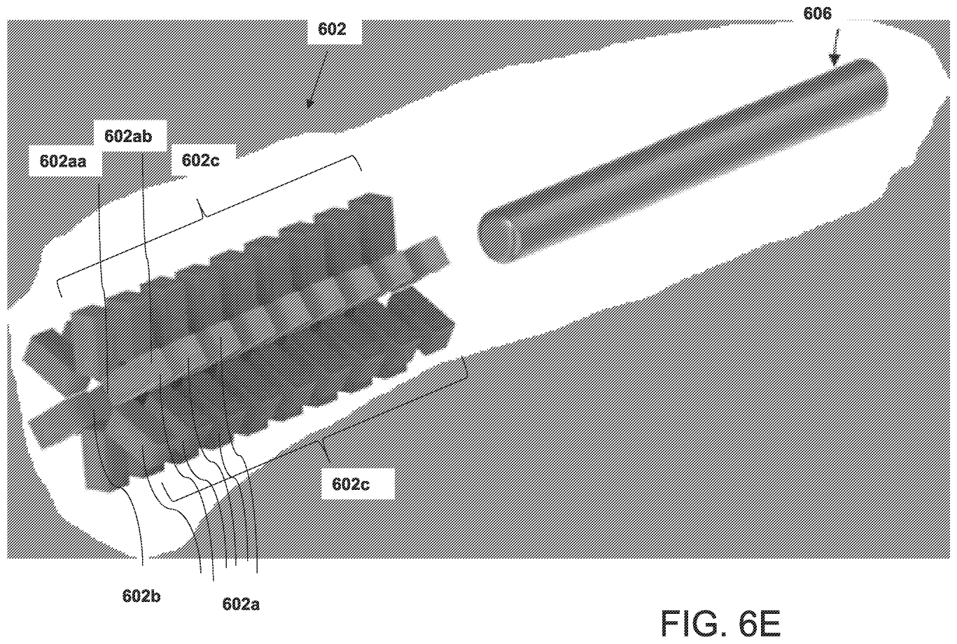

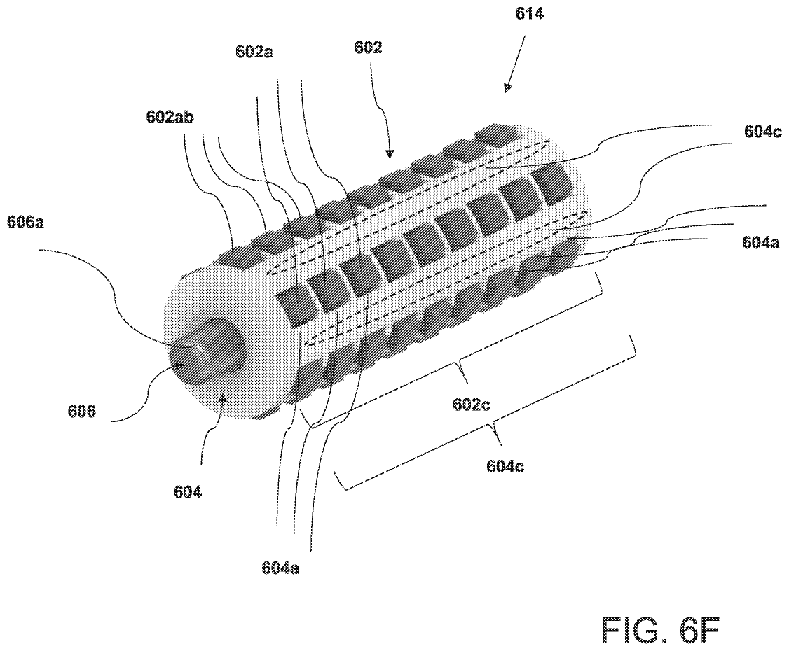

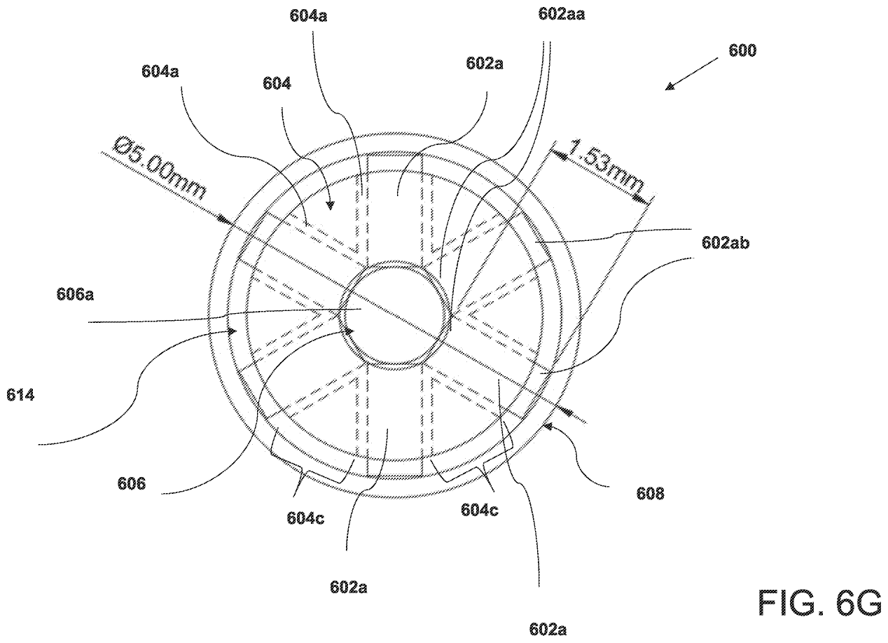

Referring now to FIGS. 6A-6G, FIGS. 6A-6G are diagrams of a non-limiting embodiment of implantable neurostimulator device 600. As shown in FIGS. 6A-6G, implantable neurostimulator device 600 may include transducer device 602, core structure 604, first electrode component 606, second electrode component 608, enclosure 610, and rectification circuit 612. In some non-limiting embodiments, implantable neurostimulator device 600 may be the same as or similar to implantable neurostimulator device 102 and/or implantable neurostimulator device 502. In some non-limiting embodiments, first electrode component 606 and/or second electrode component 608 may be the same as or similar to electrode device 306. In some non-limiting embodiments, rectification circuit 612 may be the same as or similar to rectification circuit 304.

In some non-limiting embodiments, first electrode component 606 may be sized and configured to be positioned within transducer device 602, transducer device 602 is sized and configured to be positioned within core structure 604. As shown in FIG. 6B, core structure 604 may be sized and configured to be positioned within second electrode component 608, and second electrode component 608 is sized and configured to be positioned within enclosure 610. As shown in FIG. 6C, rectification circuit 612 may be positioned at an end of enclosure 610. As further shown in FIG. 6C, rectification circuit 612 may be sized and configured to be positioned within an open end of enclosure 610. In some non-limiting embodiments, rectification circuit 612 may be positioned so that rectification circuit 612 is electrically connected to first electrode component 606 (e.g., an end of first electrode component 606 that protrudes from core structure 604, as shown in FIG. 6B). In some non-limiting embodiments, enclosure 610 may be constructed of an appropriate material, such as a biocompatible material. The biocompatible material may include titanium. In some non-limiting embodiments, enclosure 610 may have a cylindrical design. For example, enclosure 610 may have a square cylindrical design, a hexagonal cylindrical design, an octagonal cylindrical design, and/or the like.

As shown in FIG. 6D, core structure 604 may have a cylindrical design. In some non-limiting embodiments, core structure 604 may have a cylindrical design that corresponds to enclosure 610. For example, core structure 604 may have a cylindrical design that matches a shape of enclosure 610 and allows core structure 604 to be positioned within enclosure 610.

As further shown in FIG. 6D, core structure 604 may include center aperture 604b and/or a plurality of apertures 604a. In some non-limiting embodiments, center aperture 604b may be sized and configured to receive first electrode component 606. In some non-limiting embodiments, core structure 604 may be constructed based on a molding process, a mechanical process (e.g., a subtractive manufacturing process), and/or an additive manufacturing process, such as 3D printing. In some non-limiting embodiments, core structure 604 may be constructed of an appropriate material, such as a ceramic material or a partially ceramic material.

In some non-limiting embodiments core structure 604 is sized and configured to hold the plurality of transducer elements 602. In some non-limiting embodiments, a plurality of apertures 604a may be arranged in rows 604c. For example, the plurality of apertures 604a may be aligned in a straight line (e.g., extending from one end of core structure 604 to another end of core structure 604 in a straight line) to form one row 604c. In some non-limiting embodiments, core structure 604 may include a plurality of rows 604c that correspond to a number of transducer elements of transducer device 602. In some non-limiting embodiments, the plurality of rows 604c may be spaced equidistant around a perimeter (e.g., around a circumference) of core structure 604. In some non-limiting embodiments, the plurality of apertures 604a arranged in row 604c may be spaced apart. For example, the plurality of apertures 604a arranged in row 604c may be spaced equidistantly apart.

In some non-limiting embodiments, each aperture 604a of a plurality of apertures 604a may be sized and configured to receive a transducer element. In some non-limiting embodiments, a length of core structure 604 may be less than a length of enclosure 610 and/or a width (e.g., a diameter) of core structure 604 may be less than a width (e.g., a diameter) of enclosure 610. In this way, core structure 604 may be sized and configured to be positioned within enclosure 610. In some non-limiting embodiments, a length of core structure 604 may be less than or equal to a length of second electrode component 608 and/or a width (e.g., a diameter) of core structure 604 may be less than or equal to a width (e.g., a diameter) of second electrode component 608. In this way, core structure 604 may be sized and configured to be positioned within second electrode component 608.

As shown in FIG. 6E, transducer device 602 may include center aperture 602b and an array of transducer elements 602a. As further shown in FIG. 6E, center aperture 602b may be sized and configured to receive first electrode component 606. In some non-limiting embodiments, center aperture 602b of transducer device 602 is configured to be concentric with center aperture 602b of core structure 604. In some non-limiting embodiments, a plurality of transducer elements 602a may be arranged in a plurality of rows 602c. For example, the plurality of transducer elements 602a may be aligned in a straight line (e.g., extending from one end of transducer device 602 to another end of transducer device 602 in a straight line) to form one row 602c. In some non-limiting embodiments, the plurality of rows 602c are spaced equidistant around a perimeter (e.g., around a circumference) of transducer device 602. In some non-limiting embodiments, the plurality of transducer elements 602a arranged in row 602c may be spaced apart. For example, the plurality of transducer elements 602a arranged in row 602c may be spaced equidistantly apart.

As further shown in FIG. 6E, each transducer element 602a may include first end 602aa and second end 602ab. In some non-limiting embodiments, first end 602aa of transducer element 602a may be sized and configured to contact (e.g., to be electrically connected and form a conductive path) first electrode component 606. In some non-limiting embodiments, second end 602ab of transducer element 602a may be sized and configured to contact (e.g., to be electrically connected and form a conductive path) second electrode component 608.

As further shown in FIG. 6E, transducer device 602 may include a transducer array of transducer elements 602a, with six rows 602c of nine transducer elements 602a, with a total of fifty four transducer elements. In some non-limiting embodiments, first ends 602ab of transducer elements 602a extend annularly outward from a center (e.g., a center defined by center aperture 602b) of transducer device 602. In some non-limiting embodiments, the number of rows 602c and the number of transducer elements 602a may be more or less than six and fifty four, respectively, depending on the application for transducer 602 or implantable neurostimulator device 600.

As shown in FIGS. 6F and 6G, transducer assembly 614 may include transducer device 602, core structure 604, and first electrode component 606 assembled together. As further shown in FIGS. 6F and 6G, transducer device 602 may be positioned within core structure 604 and first electrode component 606 may be positioned within a center aperture of transducer device 602 and a center aperture of core structure 604. In some non-limiting embodiments, core structure 604 is sized and configured to hold the plurality of transducer elements 602a. For example, core structure 604 may have a lattice structure with a plurality of apertures 604a that are sized and configured to receive the plurality of transducer elements 602a. As further shown in FIGS. 6F and 6G, first end 606a of first electrode component 606 may protrude from core structure 604 when first electrode component 606 is positioned within a center aperture of transducer device 602 and a center aperture of core structure 604.

As further shown in FIG. 6F, a plurality of transducer elements 602a of transducer device 602 may be positioned in a plurality of corresponding apertures 604a of core structure 604. In some non-limiting embodiments, one or more rows 602c of transducer elements 602a of transducer device 602 may align with one or more rows 604c of apertures 604a of core structure 604. As further shown in FIG. 6F, second end 602ab of transducer elements 602a may protrude above a surface (e.g., an external surface) of core structure 604. As shown in FIG. 6G, first end 602aa of transducer elements 602a may be in contact with first electrode component 606. In some non-limiting embodiments, second end 602ab of transducer elements 602a may protrude above a surface (e.g., an external surface) of core structure 604 and contact second electrode component 608. In this way, an electrical path (e.g., a current path) is formed via first electrode component 606, transducer elements 602a, and second electrode component 608. Further, in this way, second end 602ab of transducer elements 602a may contact second electrode component 608 without second electrode component 606 being in contact with core structure 604.

As further shown in FIG. 6F, core structure 604 may include one or more sections 604c that are not designed to receive an acoustic signal, since there are no transducer elements 602a in section 604c. In some non-limiting embodiments, transducer device 602 may be designed to reduce the surface area of sections 604c. For example, transducer device 602 may include a number of rows 602c of transducer elements 602a that reduce the surface area of sections 604c, such as six or more (e.g., eight, ten, twelve, etc.) rows 602c. In this way, transducer device 602 may be designed to effectively receive an acoustic signal (e.g., from energy delivery device 504) despite the orientation of transducer device 602 in a body of a user.

As further shown in FIG. 6G, a length of transducer element 602a (e.g., a length of transducer element 602a as measured from first end 602aa and second end 602ab) may be 1.53 mm. In some non-limiting embodiments, a length of transducer element 602 a may be in a range of 0.5 mm to 5 mm. As further shown in FIG. 6G, a diameter of second electrode component 608 may be 5 mm. In some non-limiting embodiments, a diameter of second electrode component 608 may be in a range of 3 mm to 6 mm. In some non-limiting embodiments, a diameter of enclosure 610 may be in a range of 4 mm to 8 mm.

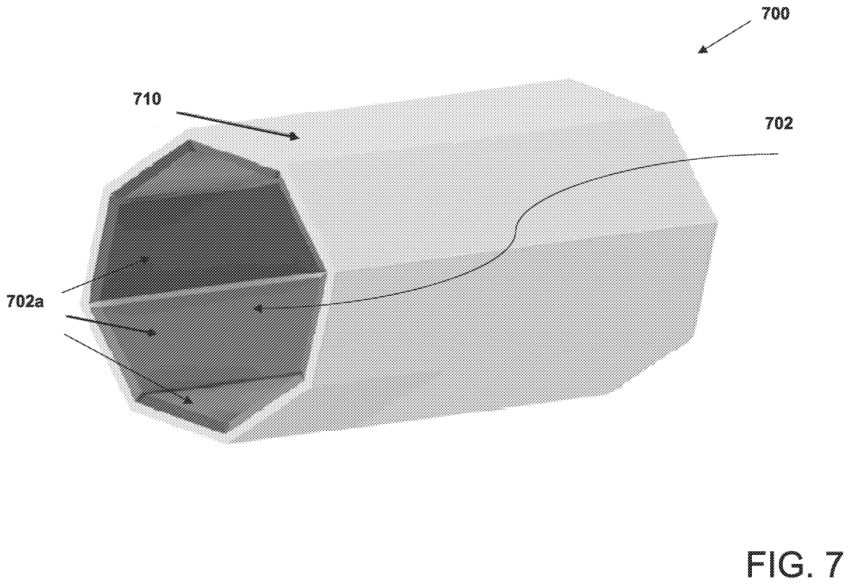

Referring now to FIG. 7, FIG. 7 is a diagram of implantable neurostimulator device 700. As shown in FIG. 7, implantable neurostimulator device 700 may include transducer device 702, which includes transducer elements 702a and enclosure 710. In some non-limiting embodiments, implantable neurostimulator device 700 may be the same as or similar to implantable neurostimulator device 600, implantable neurostimulator device 502, and/or implantable neurostimulator device 102. In some non-limiting embodiments, transducer device 702 may be the same as or similar to transducer device 602 and/or receiver device 302. In some non-limiting embodiments, enclosure 710 may be the same as or similar to enclosure 610.

As shown in FIG. 7, implantable neurostimulator device 700 may have an octagonal cylindrical design based on a shape of enclosure 710. As further shown in FIG. 7, transducer device 702 may include a plurality of transducer elements 702a that are positioned on each internal surface (e.g., each flat internal surface) of enclosure 710. In some non-limiting embodiments, one or more transducer elements 702a may include a pMUT array constructed from a thin film that is deposited onto an internal surface of enclosure 710. In some non-limiting embodiments, transducer elements 702a may include a piezoelectric micromachined ultrasonic transducer (pMUT) membrane, which is constructed with a thin film of lead zirconate titanate (PZT) and/or aluminum nitride (AlN) deposited onto a circuit (e.g., microchip). In one example, a pMUT membrane may operate in a 3-1 mode, which may allow for a thin and low profile that is well suited for a compact form factor for implantable neurostimulator device 700. In addition, performance of a pMUT membrane may be more efficient than a standard bulk piezoelectric, which may operate in a 3-3 mode. In another example, an AlN film can also have a higher receive efficiency than PZT, which may provide an advantage for transducer device 702 when used in a receive mode.

In some non-limiting embodiments, transducer elements 702a may be formed with appropriate electrode components (e.g., integral electrode components) so that separate electrode components are not necessary. The use of a pMUT array constructed from a thin film allows for enclosure 710 to have a reduced form factor, while transducer elements 702a may have received sensitivity of high bandwidth and/or lower peak frequency, without increasing the thickness of implantable neurostimulator device 700.

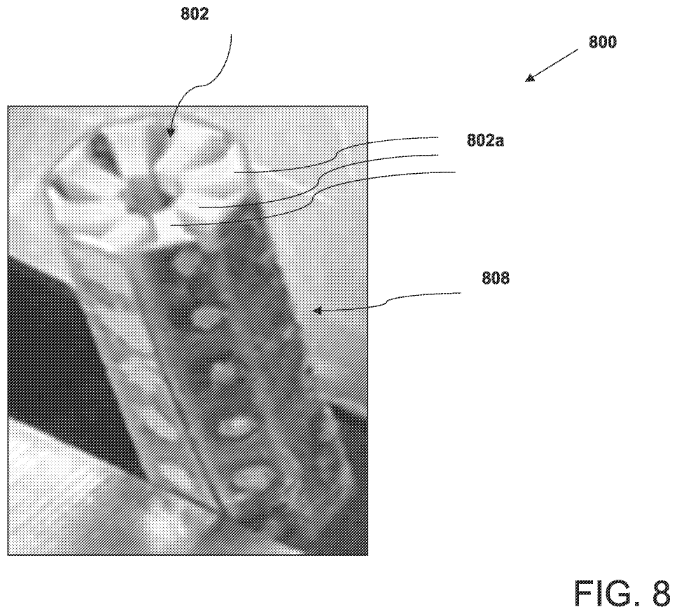

Referring now to FIG. 8, FIG. 8 is a diagram of implantable neurostimulator device 800. As shown in FIG. 8, implantable neurostimulator device 800 may include transducer device 802, which includes transducer elements 802a, and second electrode component 808. In some non-limiting embodiments, implantable neurostimulator device 800 may be the same as or similar to implantable neurostimulator device 700, implantable neurostimulator device 600, implantable neurostimulator device 502, and/or implantable neurostimulator device 102. In some non-limiting embodiments, transducer device 802 may be the same as or similar to transducer device 702, transducer device 602 and/or receiver device 302. In some non-limiting embodiments, second electrode component 808 may be the same as or similar to second electrode component 608.

In some non-limiting embodiments, electrode component 808 may include a flexible circuit (e.g., a flexible printed circuit board) with transducer elements 802a formed on a surface of electrode component 808 and electrode component 808 may be rolled into a cylindrical design as shown in FIG. 8. In some non-limiting embodiments, transducer elements 802a may include diced PZT elements. The design of implantable neurostimulator device 800 may allow for a rectification circuit to be mounted directly onto the flexible circuit, as well as a small and compact form factor with a high volume fraction of PZT for greater acoustic signal to electrical signal conversion efficiency.

Although the above embodiments have been described in detail for the purpose of illustration based on what is currently considered to be the most practical and preferred embodiments or aspects, it is to be understood that such detail is solely for that purpose and that the present disclosure is not limited to the described embodiments or aspects but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

1. An implantable neurostimulator device comprising:
at least one transducer device comprising:
a gas matrix piezoelectric (GMP) array; or
a piezoelectric micromachined ultrasound transducer (pMUT) array;
wherein the at least one transducer device comprises a material, and wherein the material comprises:
lead zirconate titanate (PZT);
single crystal lead magnesium niobate-lead titanate (PMN-PT);
aluminum nitride (AlN);
scandium (Sc) doped AlN; or
any combination thereof;
wherein the implantable neurostimulator device has a length in a range of 5 mm to mm and a diameter in a range of 1 mm to 5 mm.

2. The implantable neurostimulator device of claim 1, wherein the at least one transducer device comprises a plurality of transducer elements, and wherein each transducer element of the plurality of transducer elements comprises:
PZT;
polyvinylidene fluoride;
polyvinylidene difluoride (PVDF); or
any combination thereof.

3. The implantable neurostimulator device of claim 2, wherein the plurality of transducer elements are formed in a plurality of rows, and wherein the plurality of rows are spaced equidistant around a perimeter of the at least one transducer device.

4. The implantable neurostimulator device of claim 1, wherein the at least one transducer device comprises the pMUT array, and wherein the pMUT array comprises:
PZT; and
AlN.

5. The implantable neurostimulator device of claim 1, wherein the at least one transducer device is operated independent of an onboard processor.

6. The implantable neurostimulator device of claim 1, further comprising:
an enclosure, wherein the enclosure has a cylindrical design with a plurality of internal surfaces; and
wherein the at least one transducer device comprises a plurality of elements of the pMUT array that are positioned on the plurality of internal surfaces of the enclosure.

7. The implantable neurostimulator device of claim 6, wherein the plurality of elements of the pMUT array comprises a plurality of thin film elements deposited on the plurality of internal surfaces of the enclosure.

8. The implantable neurostimulator device of claim 1, wherein the at least one transducer device is configured to:
receive an acoustic signal from an energy delivery device; and
convert the acoustic signal to an electrical signal to provide an action potential to activate a nerve.

9. The implantable neurostimulator device of claim 8, further comprising:
an electrode device, wherein the electrode device is configured to receive the electrical signal and apply the electrical signal to an environment inside a body of a patient.

10. The implantable neurostimulator device of claim 1, further comprising:
a rectification circuit, wherein the rectification circuit is configured to covert an output of the at least one transducer to a direct current (DC) electrical pulse to provide electrical stimulation to an environment inside a body of a patient.

11. The implantable neurostimulator device of claim 1, wherein the at least one transducer device comprises a plurality of transducer elements, and the implantable neurostimulator device further comprises:
a core structure, wherein the core structure is sized and configured to hold the plurality of transducer elements.

12. A system for providing neurostimulation to a target location of a body of a user, comprising:
an implantable neurostimulator device comprising:
at least one first transducer device comprising:
a gas matrix piezoelectric (GMP) array; or
a piezoelectric micromachined ultrasound transducer (pMUT) array;
wherein the at least one first transducer device comprises a material, and wherein the material comprises:
lead zirconate titanate (PZT);
single crystal lead magnesium niobate-lead titanate (PMN-PT);
aluminum nitride (AlN);
scandium (Sc) doped AlN; or
any combination thereof;
wherein the implantable neurostimulator device has a length in a range of 5 mm to 20 mm and a diameter in a range of 1 mm to 5 mm;
an energy delivery device comprising:
at least one second transducer device, wherein the at least one second transducer device comprises a material, and wherein the material comprises:
lead zirconate titanate (PZT);
polyvinylidene difluoride (PVDF);
aluminum nitride (AlN);
scandium (Sc) doped AlN; or
any combination thereof;
wherein the at least one second transducer device comprises:
a gas matrix piezoelectric (GMP) array;
a polymer matrix piezocomposite array;
a capacitive micro-machined acoustic transducer (cMUT) array; or
a piezoelectric micromachined ultrasound transducer (pMUT) array; and
at least one beam-forming processor configured to:
cause the at least one second transducer device to produce an acoustic wave;
wherein the implantable neurostimulator device is configured to:
receive the acoustic wave from the energy delivery device;
covert the acoustic wave to an electrical energy signal; and
apply the electrical energy signal to an environment inside a body of a patient.

13. The system of claim 12, wherein the acoustic wave comprises a plurality of pulses having a pulse repetition frequency, wherein a pulse width of each pulse of the plurality of pulses is in a range of 10 ns to 10 μs, and wherein the pulse repetition frequency is in a range of 1 Hz to 50 Hz.

14. The system of claim 12, wherein the at least one first transducer device comprises a plurality of transducer elements, and wherein each transducer element of the plurality of transducer elements comprises:
PZT;
polyvinylidene fluoride;
PVDF; or
any combination thereof.

15. The system of claim 12, wherein the plurality of transducer elements are formed in a plurality of rows, and wherein the plurality of rows are spaced equidistant around a perimeter of the at least one first transducer device.

16. The system of claim 12, wherein the at least one first transducer device comprises the pMUT array, and wherein the pMUT array comprises:
PZT; and
AlN.

17. The system of claim 12, wherein the at least one first transducer device is operated independent of an onboard processor.

18. The system of claim 12, further comprising:
an enclosure, wherein the enclosure has a cylindrical design with a plurality of internal surfaces; and
wherein the at least one first transducer device comprises a plurality of elements of the pMUT array that are positioned on the plurality of internal surfaces of the enclosure.

19. The system of claim 18, wherein the plurality of elements of the pMUT array comprises a plurality of thin film elements deposited on the plurality of internal surfaces of the enclosure.

20. The system of claim 12, wherein the at least one first transducer device is configured to:
receive an acoustic signal from an energy delivery device; and
convert the acoustic signal to an electrical signal to provide an action potential to activate a nerve.

21. The system of claim 20, further comprising:
an electrode device, wherein the electrode device is configured to receive the electrical signal and apply the electrical signal to an environment inside a body of a patient.

22. The system of claim 12, further comprising:
a rectification circuit, wherein the rectification circuit is configured to covert an output of the at least one first transducer to a direct current (DC) electrical pulse to provide electrical stimulation to an environment inside a body of a patient.

23. The system of claim 12, wherein the at least one first transducer device comprises a plurality of transducer elements, and the implantable neurostimulator device further comprises:
a core structure, wherein the core structure is sized and configured to hold the plurality of transducer elements.

24-35. (canceled)
